(19) United States
(12) Patent Application Publication
(10) Pub. No.: US 2008/0234493 A1
(43) Pub. Date: Sep. 25, 2008

(54) Process for the Preparation of 2-[N-[{S)-1-Ethoxycarbonyl-3-Phenylpropyl]-(S)-Alanyl]-(1S,3S,5S)-2-Azabicyclo[3.3.0]Oct- an-3-Carboxylic Acid

(75) Inventors: Krzysztof Bankowski, Warszawa (PL); Teresa Wachal, Warszawa (PL); Katarzyna Sidoryk, Warszawa (PL); Wieslaw Szelejewski, Warszawa (PL)

Correspondence Address: MATTHIAS SCHOLL, 14781 MEMORIAL DRIVE, SUITE 1319, HOUSTON, TX, 77079, US

(73) Assignee: INSTYTUT FARMACEUTYCZNY, WARSZAWA (PL)

(21) Appl. No.: 11/568,922

(22) PCT Filed: May 12, 2005
(86) PCT No.: PCT/PL05/00029

§ 371 Date: Apr. 30, 2007

(30) Foreign Application Priority Data
May 12, 2004 (PL) P-367931

(51) Int. Cl. C07D 209/02

(52) U.S. Cl. 548/452

(57) ABSTRACT

A process for preparation of 2-[N-[(S)-1-ethoxycarbonyl-3-phenylpropyl]-(S)-alanyl]-(1S,3S,5S)-2- azabicyclo [3.3.0] octane-3-carboxylic acid, ie. Ramipril, involves condensation of an activated derivative of 2-[N-](S)-1-ethoxycarbonyl-3-phenylpropyl]-(S)-alanine with racemic (1R*,3R*,5R*)-2-azabicyclo[3.3.0]octane-3-carboxylic acid, and then the desired diastereoisomer (1a) is separated from the obtained diastereoisomeric mixture of 2-[N-[(S)-1-ethoxycarbonyl-3-phenylpropyl](S)-alanyl]-(1S,3S,5S)-2-azabicyclo[3.3.0]octane-3-carboxylic acid (1a) and 2-[N-[(S)-1-ethoxycarbonyl-3-phenylpropyl -alanyl]-(1R,3R,5R)-2-azabicyclo[3.3.0]octane-3-carboxylic acid (1b) by treating it with a solvent that selectively dissolves the undesired diastereoisomer (1b) while the diastereoisomer (1a) remains undissolved.

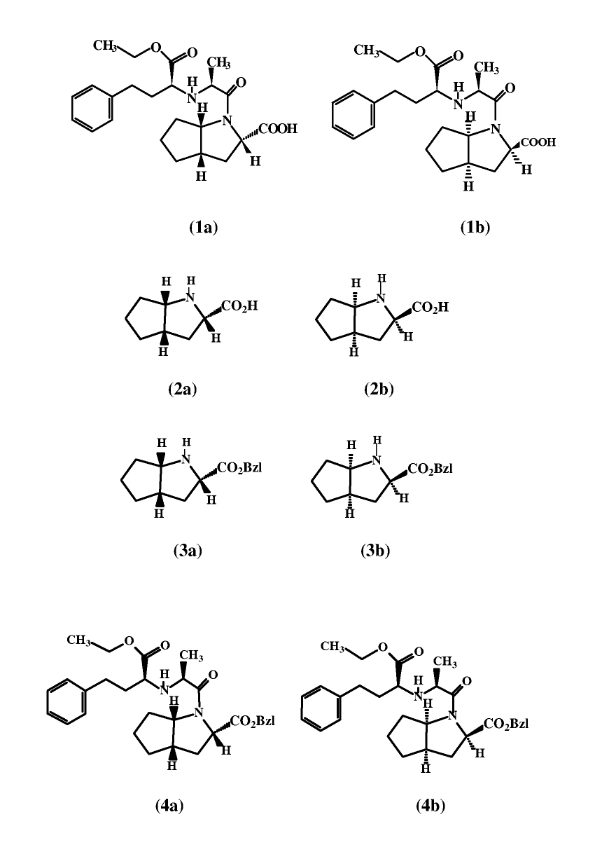

The invention relates to a process for the preparation of 2-[N-[(S)-1-ethoxycarbonyl-3-phenylpropyl]-(S)-alanyl]-(1S,3S,5S)-2-azabicyclo[3.3.0]octane-3-carboxylic acid.

2-[N-[(S)-1-ethoxycarbonyl-3-phenylpropyl]-(S)-alanyl]-(1S,3S,5S) -2-azabicyclo[3.3.0]octane-3-carboxylic acid, otherwise known under the international non-proprietary name (INN) Ramipril. Ramipril is one of angiotensin converting enzyme (ACE) inhibititors that are a large group of drugs widely used in treating arterial hypertension. Ramipril is used both in mono-therapy and in combined therapy of hypertension, disorders of the coronary arteries, in heart failure therapy, in particular in post-myocardial infarction failure.

Ramipril, the chemical structure of which is represented by the formula (1a),

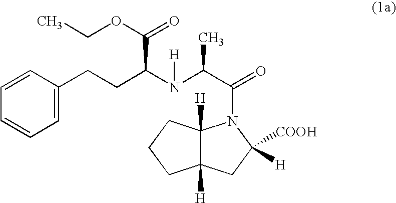

is an acylated derivative of (1S,3S,5S)-2-azabicyclo[3.3.0]octane-3-carboxylic acid, where the acyl group is derived from N-[1(S)-(ethoxycarbonyl)-3-phenylpropyl]-(S)-alanine.

In practice, the synthesis of Ramipril is based upon two strategies.

According to one of these strategies, the starting material is racemic (1R*,3R*,5R*) -2-azabicyclo[3.3.0]octane-3-carboxylic acid, otherwise known as (1)-endo,cis-2-azabicyclo[3.3.0]octane-3-carboxylic acid, being a mixture of diastereoisomers described by the formulae (2a)/(2b)

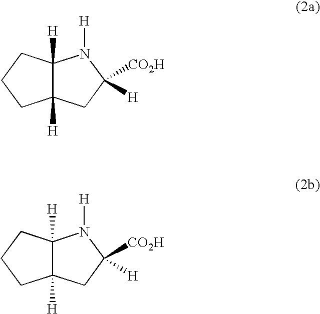

that is converted to the racemic benzyl esters (a mixture of (3a) and (3b)).

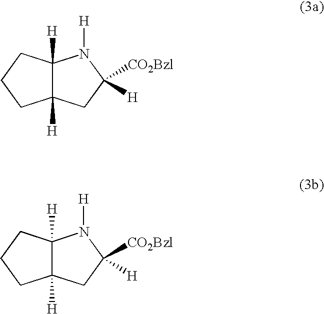

Next, the esters are condensed with N-[1(S)-(ethoxycarbonyl)-3-phenylpropyl]-(S)-alanine by any method known in the chemistry of peptides.

The mixture of diastereoisomeric benzyl esters (4a) and (4b):

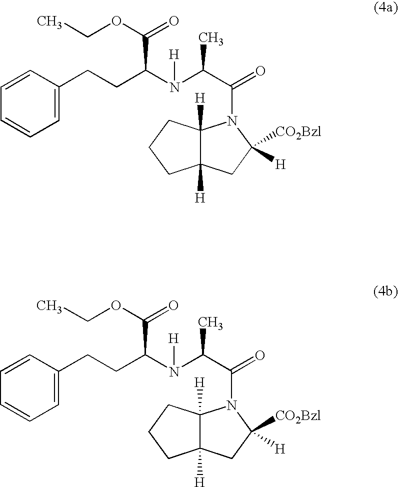

obtained as above is separated by column chromatography on silica gel. The Ramipril benzyl ester (4a) is then converted by catalytic hydrogenation into Ramipril. This approach has been first disclosed in the description of the European patent EP 0 007,902, and later in the description of the patents EP 0 050,800, ES 459,251, in the description of the Chinese patent CN 11,006,386 and in publications of Taetz V., Geiger R., Henning R., Urbach; Arzneim.-Forsch./Drug Res., 1984, 34(II), No. 10b, 1399 and Taetz V., Geiger R., Gaul H., Tetr. Letters, 25(40), 4479-4482.

A disadvantage of this method, in particular considering its industrial scale application is the necessity of protecting the carboxylic group of (1R*,3R*,5R*) -2-azabicyclo[3.3.0]octane-3-carboxylic acid as the benzyl ester and, mainly, a separation of diastereoisomeric benzyl esters by column chromatography that is very time-consuming, costly and difficult for scaling-up. Furthermore, the strategy imposes the necessity of cleaving the benzyl ester moiety by catalytic hydrogenation in the final stage of the synthesis.

According to the second strategy, N-[1(S)-(ethoxycarbonyl)-3-phenylpropyl]-(S)-alanine is condensed with the optically pure benzyl ester of (1S,3S,5S)-2-azabicyclo[3.3.0]octane-3-carboxylic acid (3a). The product of the condensation, Ramipril benzyl ester (4a), is then catalytically reduced to Ramipril (1a). A disadvantage of this approach is the necessity of gaining the benzyl ester (3a) of a high optical purity.

The benzyl ester (3a) could be obtained either by separation of the racemate (3a)/(3b) or by an enantioselective synthesis of (1S,3S,5S)-2-azabicyclo[3.3.0]octane-3-carboxylic acid (2a) and subsequent esterification of the acid obtained.

There are literature references concerning separation of the racemic benzyl esters (3a)/(3b) by preparation and subsequent fractional crystallization of diastereoisomeric salts obtained a) with aromatic N-benzyloxycarbonyl-L-amino acids, e.g. N-benzyloxycarbonyl-L-phenylalanine (EP 0 115,345; Taetz V. et al., Arzneim.-Forsch./Drug Res., 1984,34(II), No. 10b, 1399; Taetz V., Geiger R., Gaul H., Tetr. Letters, 25, 40, 4479-4482), b) with (S)-mandelic acid (Martens J. et. al., Journal of Prakt. Chemie, 332, (6), 1990, 1111-1117), or c) with dibenzoyl-L-tartaric acid (EP 0 146,080). A shortcoming of all these methods is a low yield of separation and the necessity of using expensive, optically active separating agents.

On the other hand, overall yields of (1S,3S,5S)-2-azabicyclo[3.3.0]octane-3-carboxylic acid (2a) obtained by the methods independently proposed by Harward et al. (Tetrahedron Letters, 1993, 34 (41), 6603-6606) and Urbach H. et al. (Heterocycles, 28, 2, 957-965, 1989) are 13% and 5.5%, respectively, which precludes application of any of the methods in the industry.

The specification of the U.S. Pat. No. 6,407,262 discloses a method of separating the mixture of Ramipril diastereoisomers (1a) and (1b)

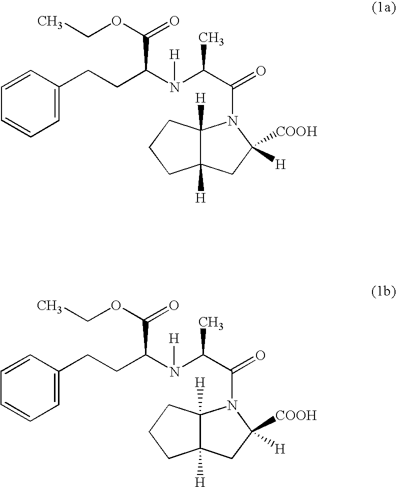

by using a solvent that precipitates the pure diastereoisomer (1a). A process involving crystallization of the diastereoisomer (1a) from an ethyl acetate solution by adding diisopropyl ether as an anti-solvent is disclosed in the examples. Other solvents that could be used for this purpose comprise used for this purpose comprise isopropyl acetate, butyl acetate and ethyl propionate. The disclosed method requires seeding of the reaction mixture with crystals of pure Ramipril. Yields of separation vary from 40% to 60%. The authors of U.S. Pat. No. 6,407,262 state that the mixture of the diastereoisomers (1a/1b) is prepared by methods from the prior art, but any particular method is not specified. Moreover, the teaching of said patent description suggests that the synthetic route involves the use of diastereoisomeric benzyl esters. The suggestion is supported by the proposed method of isolating of the pure benzyl ester of Ramipril as the maleate salt.

The third, potentially useful strategy for synthesizing ACE inhibitors, is based upon direct condensation of various activated derivatives of N-[1(S)-(ethoxycarbonyl)-3-phenylpropyl]-(S)-alanine with appropriate enantiomer of a free amino acid or a salt thereof. Up to now, the method was used mainly for preparation of enalapril (cf. specifications of the patents U.S. Pat. No. 5,977,380, DE 19721290, ES 2156050). Although the possibility of producing Ramipril by using (1S,3S,5S)-2-azabicyclo[3.3.0]octane-3-carboxylic acid as the starting material was suggested either in the specifications or in claims, it has not been supported by any synthetic procedure. So far, no racemic amino acid has been used as a starting material in condensations of this type, presumably due to the necessity of separating two diastereoisomeric products in the next steps.

Now, due to the inventing of a simple method of isolating the desired diastereoisomer (1a) from the diastereoisomeric mixture of 2-[N-[(S)-1-ethoxycarbonyl-3-phenylpropyl]-(S)-alanyl]-(1S,3S,5S)-2-azabicyclo[3.3.0]octane-3-carboxylic acid (1a) and 2-[N-[(S) -1-ethoxycarbonyl-3-phenylpropyl]-(S)-alanyl]-(1R,3R,5R) -2-azabicyclo[3.3.0]octane-3-carboxylic acid (1b), the strategy of activated derivatives has been successfully applied for production of Ramipril using racemic (1R*,3R*,5R*)-2-azabicyclo[3.3.0]octane-3-carboxylic acid that is more readily available than the single optical isomer.

The present invention provides the process for the preparation of 2-[N-[(S)-1-ethoxycarbonyl-3-phenylpropyl]-(S)-alanyl]-(1S,3S,5S)-2-azabicyclo[3.3.0]octane-3-carboxylic acid, i.e. Ramipril (1a)

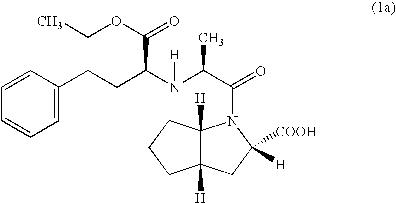

by condensation of an activated derivative of N-[(S)-1-ethoxycarbonyl-3-phenylpropyl]-(S)-alanine with a racemic (1R*,3R*,5R*)-2-azabicyclo[3.3.0]octane-3-carboxylic acid (2a/2b),

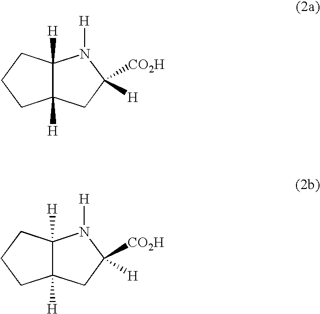

followed by the isolation of the desired diastereoisomer (1a) from the obtained diastereoisomeric mixture of 2-[N-[(S)-1-ethoxycarbonyl-3-phenylpropyl]-(S)-alanyl]-(1S,3S,5S) -2-azabicyclo[3.3.0]octane-3-carboxylic acid (1a) and 2-[N-[(S)-1-ethoxycarbonyl-3-phenylpropyl]-(S)-alanyl]-(1R,3R,5R)-2-azabicyclo[3.3.0]octane-3-carboxylic acid (1b),

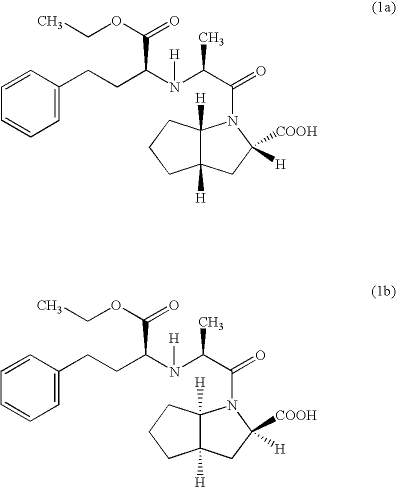

by treating the mixture with a solvent that selectively dissolves the undesired diastereoisomer (1b) while the diastereoisomer (1a) remains undissolved.

The starting compound, N-[(S)-1-ethoxycarbonyl-3-phenylpropyl]-(S)-alanine, could be activated and coupled with amino acid by any method known to those skilled in the art, for example by activated ester method (nitro- and chloro-phenyl, succinimidyl, and the like), carbodiimide method (DCC/HOBt, and the like), mixed anhydride method, or using another peptide coupling reagents as uronium/phosphonium-based compounds (TBTU, HBTU, PyBOP, and the like).

In the preferred embodiment of the invention N-[(S)-1-ethoxycarbonyl-3-phenylpropyl]-(S)-alanine is converted to its N-carboxyanhydride derivative. Alternatively, an activated ester of N-[(S)-1-ethoxycarbonyl-3-phenylpropyl]-(S)-alanine, preferably a succinimidyl ester could be used.

The condensation of 2-[N-[(S)-1-ethoxycarbonyl-3-phenylpropyl]-(S)-alanine-N-carboxyanhydride with free racemic (1R*,3R*,5R*)-2-azabicyclo[3.3.0]octane-3-carboxylic acid involves neutralization in situ of the corresponding hydrochloride by adding a base, preferably a tertiary amine, most preferably triethylamine. The reaction is performed in an aprotic solvent, preferably in dichloromethane or in dimethylformamide.

The type of the base chosen for neutralizing the hydrochloride and the method of activating 2-[N-[(S)-1-ethoxycarbonyl-3-phenylpropyl]-(S)-alanine are not limiting the scope of the present invention.

The oily diastereoisomeric mixture of 2-[N-[(S)-1-ethoxycarbonyl-3-phenylpropyl]-(S)-alanyl]-(1S,3S,5S)-2-azabicyclo[3.3.0]octane-3-carboxylic acid and 2-[N-[(S)-1-ethoxycarbonyl-3-phenylpropyl]-(S)-alanyl]-(1R,3R,5R)-2-azabicyclo[3.3.0]octane-3-carboxylic acid, obtained by condensation is treated at room temperature with such amount of a solvent that is sufficient for dissolving the undesired diastereoisomer.

Readily available ethyl acetate is an appropriate solvent for this purpose. Unexpectedly, on applying the method according to the invention it has been found that if ethyl acetate is used, then seeding of the reaction mixture with the desired diastereoisomer or addition of any non-solvent is not necessary. After several minutes, crystalline Ramipril precipitates from the ethyl acetate solution, while the undesired diastereoisomer remains therein due to its good solubility in the solvent. Chemical and stereochemical purity of the obtained Ramipril is controlled by high performance liquid chromatography (HPLC). A pharmaceutical substance, purity of which exceeds 99.0% (HPLC) is obtained with approximately 50% yield (calculated on the content of (1S*,3S*,5S*)-enantiomer in racemic (1R*,3R*,5R*) -2-azabicyclo[3.3.0]octane-3-carboxylic acid used as the starting material). If needed, the product could be treated with much larger volume of ethyl acetate and then filtered to remove any potential impurities.

The invention allows to obtain Ramipril from a racemic (1R*,3R*,5R*)-2-azabicyclo[3.3.0]octane-3-carboxylic acid that is a starting material more readily available than its single stereoisomer and from commercially available [N-[(S)-1-ethoxycarbonyl-3-phenylpropyl]-(S)-alanyl-N-carboxyanhydride.

The present invention provides an efficient and economical process for the preparation of the mixture of Ramipril (1a) and its diastereoisomer (1b) starting directly from a racemic (1R*,3R*,5R*)-2-azabicyclo[3.3.0]octane-3-carboxylic acid, said process ensures a technologically simple method for isolating of Ramipril that does not require seeding of the reaction mixture with the crystals of the desired diastereoisomer.

In the process according to the invention, the number of steps necessary to perform the synthesis is significantly reduced by eliminating preparation of diastereoisomeric benzyl esters, separation of the esters by column chromatography and catalytic, reductive cleavage of the benzyl ester moiety (as compared to the method defined as the first strategy).

Moreover, the process according to the invention allows to avoid a multi-stage and expensive separation of racemic starting materials into enantiomers that is a necessary operation in the second strategy. Separation of diastereoisomers is accomplished by taking advantage of solubility difference between Ramipril and its diastereoisomer.

The invention is further illustrated by the following examples that do not limit its scope.

### EXAMPLE 1

Preparation of the mixture of 2-[N-[(S)-1-ethoxycarbonyl-3-phenylpropyl]-(S)-alanyl]-(1S,3S,5S)-2-azabicyclo[3.3.0]octane-3-carboxylic acid (1a) and 2-[N-[(S)-1-ethoxycarbonyl-3-phenylpropyl]-(S)-alanyl]-(1R,3R,5R)-2-azabicyclo[3.3.0]octane-3-carboxylic acid (1b).

Racemic (1R*,3R*,5R*)-2-azabicyclo[3.3.0]octane-3-carboxylic acid hydrochloride (10.35 g, 33.9 mM) was suspended with stirring in 70 mL of methylene chloride and then triethylamine (4.71 ml, 33.9 mM) was added at room temperature followed by N-[(S)-1-ethoxycarbonyl-3-phenylpropyl]-(S)-alanine-N-carboxyanhydride (10.3 g, 33.9 mM). The suspension was stirred overnight at room temperature (TLC shows completion of the reaction). The precipitate was filtered, washed with 20 mL of methylene chloride and the filtrate along with the washings were evaporated in vacuo to afford an oily mixture (13.1 g) containing Ramipril (5a) and its diastereoisomer (5b). [TLC (chloroform:methanol:acetic acid=10:0.8:0.2); R_{f} (5a)=0.43, R_{f} (5b)=0.56].

### EXAMPLE 2

Isolation of Ramipril from a mixture of 2-[N-[(S)-1-ethoxycarbonyl-3-phenylpropyl]-(S)-alanyl]-(1S,3S,5S)-2-azabicyclo[3.3.0]octane-3-carboxylic acid (1a) and 2-[N-[(S)-1-ethoxycarbonyl-3-phenylpropyl]-(S)-alanyl]-(1R,3R,5R)-2-azabicyclo[3.3.0]octane-3-carboxylic acid (1b)

The oil, obtained as in Example 1 (13.6 g) was treated at room temperature with 20 mL of ethyl acetate. A part of the oil was dissolved upon stirring the mixture for several minutes and a crystalline precipitate has appeared. The whole mixture was left for 8 hours at approx. 5° C. to complete the crystallization. The precipitate was filtered and washed on the filter with ethyl acetate (3×20 mL) and dried in the air to afford 3.46 g of Ramipril (yield 49%, calculated on the content of the (1S*,3S*,5S*)-enantiomer in racemic (1R*,3R*,5R*)-2-azabicyclo[3.3.0]octane-3-carboxylic acid used as the starting material).

Purity by HPLC-RP: >99%, content of the other diastereoisomer: 0-0.4%; R_{f}=0.53 (CHCl₃:CH₃OH:AcOH=10:0.8:0.2); [α]²²_{D}=+37.3° [c1, (14 mL conc. HCl/86 mL MeOH)]; IR (KBr): 500, 756, 1065, 1187, 1375, 1464, 1653, 1743, 2937, 3068, 3281 cm⁻¹.

1-20. (canceled)

21. A process for the preparation of 2-[N-[(S)-1-ethoxycarbonyl-3-phenylpropyl]-(S)-alanyl]-(1S,3S,5S)-2-azabicyclo[3.3.0]octane-3-carboxylic acid (1a), 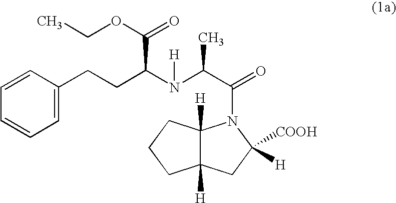
characterized in that an activated derivative of N-[(S)-1-ethoxycarbonyl-3-phenylpropyl]-(S)-alanine is condensed with a racemic (1R*,3R*,5R*)-2-azabicyclo[3.3.0]octane-3-carboxylic acid (2a/2b), 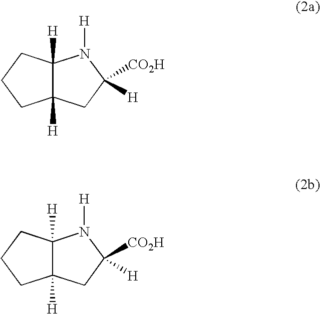
and then the desired diastereoisomer (1a) is separated from the obtained diastereoisomeric mixture of 2-[N-[(S)-1-ethoxycarbonyl-3-phenylpropyl]-(S)-alanyl]-(1S,3S,5S)-2-azabicyclo[3.3.0]octane-3-carboxylic acid (1a) and 2-[N-[(S)-1-ethoxycarbonyl-3-phenylpropyl]-(S)-alanyl]-(1R,3R,5R)-2-azabicyclo[3.3.0]octane-3-carboxylic acid (1b) 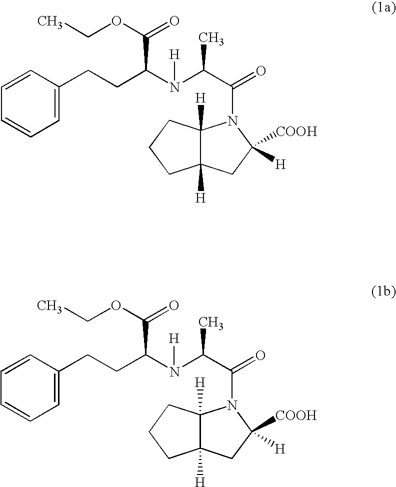
by treating the mixture with a solvent that selectively dissolves the undesired diastereoisomer (1b) while the diastereoisomer (1a) remains undissolved.

22. The process according to claim 21 in which the solvent that selectively dissolves the undesired diastereoisomer (1b) is ethyl acetate.

23. The process according to claims 21 in which the activated derivative of N-[(S)-1-ethoxycarbonyl-3-phenylpropyl]-(S)-alanine is N-carboxyanhydride thereof.

24. The process according to claims 21 in which the activated derivative of N-[(S)-1-ethoxycarbonyl-3-phenylpropyl]-(S)-alanine is the succinylimidyl ester thereof.

25. The process according to claim 21 in which 2-[N-[(S)-1-ethoxycarbonyl-3-phenylpropyl]-(S)-alanyl]-(1S,3S,5S)-2-azabicyclo[3.3.0]octane-3-carboxylic acid is isolated by filtration.

26. The process according to claims 22 in which the activated derivative of N-[(S)-1-ethoxycarbonyl-3-phenylpropyl]-(S)-alanine is N-carboxyanhydride thereof.

27. The process according to claim 22 in which the activated derivative of N-[(S)-1-ethoxycarbonyl-3-phenylpropyl]-(S)-alanine is the succinylimidyl ester thereof.

28. A method for separating out 2-[N-[(S)-1-ethoxycarbonyl-3-phenylpropyl]-(S)-alanyl]-(1S,3S,5S)-2-azabicyclo[3.3.0]octane-3-carboxylic acid (1a) from a diastereoisomeric mixture comprising 2-[N-[(S)-1-ethoxycarbonyl-3-phenylpropyl]-(S)-alanyl]-(1S,3S,5S)-2-azabicyclo[3.3.0]octane-3-carboxylic acid (1a) and 2-[N-[(S)-1-ethoxycarbonyl-3-phenylpropyl]-(S)-alanyl]-(1R,3R,5R)-2-azabicyclo[3.3.0]octane-3-carboxylic acid (1b) 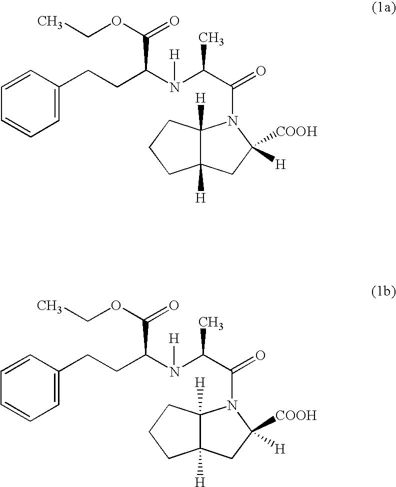
comprising
adding to said mixture a solvent that selectively dissolves diastereoisomer (1b) while the diastereoisomer (1a) remains undissolved.

29. The method of claim 28 wherein said solvent is ethyl acetate.

30. The method of claim 28 wherein upon addition of said solvent to said diastereoisomeric mixture, 2-[N-[(S)-1-ethoxycarbonyl-3-phenylpropyl]-(S)-alanyl]-(1S,3S,5S)-2-azabicyclo[3.3.0]octane-3-carboxylic acid (1a) crystallizes out of the mixture.

31. The method of claim 30 wherein said 2-[N-[(S)-1-ethoxycarbonyl-3-phenylpropyl]-(S)-alanyl]-(1S,3S,5S)-2-azabicyclo[3.3.0]octane-3-carboxylic acid (1a) is filtered off.

32. The method of claim 28 wherein no seeding of the diastereoisomeric mixture with crystals of pure 2-[N-[(S)-1-ethoxycarbonyl-3-phenylpropyl]-(S)-alanyl]-(1S,3S,5S)-2-azabicyclo[3.3.0]octane-3-carboxylic acid (1a) is necessary.

33. The method of claim 30 wherein after addition of said solvent, the mixture is allowed to stand for six to twelve hours at a temperature of between about −10° C. to about +10° C. to complete the crystallization.

34. The method of claim 31 wherein after said 2-[N-[(S)-1-ethoxycarbonyl-3-phenylpropyl]-(S)-alanyl]-(1S,3S,5S)-2-azabicyclo[3.3.0]octane-3-carboxylic acid (1a) is filtered off, it is washed with ethyl acetate and allowed to dry in the air.

35. The method of claim 28 wherein said solvent is added in such amount as is sufficient for dissolving one of the diastereomers while not dissolving the other diastereomer.

36. The method of claim 21 wherein the ratio of said diastereomeric mixture to said solvent is from about 0.25 g/mL to about 2 g/mL.

37. A method of synthesis of a diastereomeric mixture of 2-[N-[(S)-1-ethoxycarbonyl-3-phenylpropyl]-(S)-alanyl]-(1S,3S,5S)-2-azabicyclo[3.3.0]octane-3-carboxylic acid (1a) and 2-[N-[(S)-1-ethoxycarbonyl-3-phenylpropyl]-(S)-alanyl]-(1R,3R,5R)-2-azabicyclo[3.3.0]octane-3-carboxylic acid (1b) 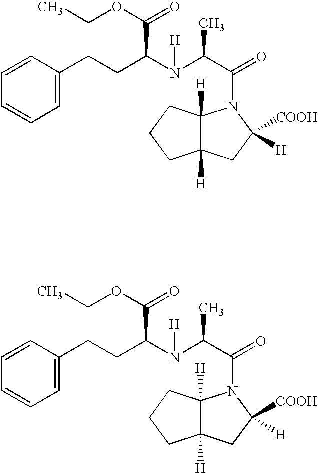
comprising
(a) activating N-[(S)-1-ethoxycarbonyl-3-phenylpropyl]-(S)-alanine to obtain an activated derivative of N-[(S)-1-ethoxycarbonyl-3-phenylpropyl]-(S)-alanine; and
(b) condensing of the said activated derivative of N-[(S)-1-ethoxycarbonyl-3-phenylpropyl]-(S)-alanine with a racemic (1R*,3R*,5R*)-2-azabicyclo[3.3.0]octane-3-carboxylic acid (2a/2b)

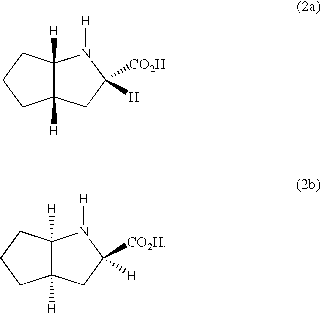

38. The method of claim 37, wherein
in (a), N-[(S)-1-ethoxycarbonyl-3-phenylpropyl]-(S)-alanine is activated via an activated ester method, a carbodiimide method, a mixed anhydrides method or any other peptide coupling method; and
in (b), the condensation reaction is conducted in an aprotic solvent to which a base is added.

39. The method of claim 37, wherein said activated derivative is N-[(S)-1-ethoxycarbonyl-3-phenylpropyl]-(S)-alanine-N-carboxyanhydride.

40. The method of claim 37 comprising reacting N-[(S)-1-ethoxycarbonyl-3-phenylpropyl]-(S)-alanine-N-carboxyanhydride with a racemic (1R*,3R*,5R*)-2-azabicyclo[3.3.0]octane-3-carboxylic acid (2a/2b) 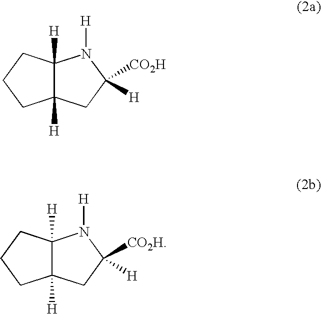
in dichloromethane or dimethylformamide in the presence of triethylamine.
